(19) United States
(12) Patent Application Publication
(10) Pub. No.: US 2026/0161020 A1
(43) Pub. Date: Jun. 11, 2026

(54) MOBILE ACQUISITION OF IMAGE CAPTURE DATA FOR SPECTACLE LENSES

(71) Applicant: Hans-Joachim OLLENDORF, Tangerhütte (DE)

(72) Inventor: Hans-Joachim OLLENDORF, Tangerhütte (DE)

(21) Appl. No.: 19/413,872

(22) Filed: Dec. 9, 2025

(30) Foreign Application Priority Data
Dec. 11, 2024 (DE) 10 2024 137 124.2

(51) Int. Cl. G02C 13/00; H04N 13/296; H04N 23/56; H04N 23/63; H04N 23/90

(52) U.S. Cl. CPC G02C 13/005; H04N 13/296; H04N 23/56; H04N 23/63; H04N 23/90

(57) ABSTRACT

A method for mobile acquisition of image capture data for adapting spectacle lenses to a spectacle frame is disclosed. A mobile computer-based terminal device with a touchscreen monitor and a stereo camera system having at least two spaced-apart electronic cameras captures the facial area of a test person wearing a properly fitted spectacle frame. The cameras simultaneously generate synchronous images by being triggered in a clock-synchronous manner through the terminal device. Triggering is performed using a clock signal and an image trigger signal, or using a clock signal, an image trigger signal, and a flash trigger signal. The synchronized image data enables determination of spectacle lens parameters for lens adaptation.

### CROSS REFERENCE TO RELATED APPLICATIONS

This patent application claims priority to and the benefit of German patent application 10 2024 137 124.2, filed on Dec. 11, 2024, which is hereby incorporated by reference in its entirety.

### TECHNICAL FIELD

This disclosure relates to a method for the mobile acquisition of image capture data for adjusting spectacle lenses to a spectacle frame and a mobile capturing system.

### BACKGROUND

Vision correction using spectacle lenses requires precise alignment of the lenses with the user's eyes, accounting for both the user's physiognomy and the geometry of the selected spectacle frame. To ensure optimal visual correction, image capture data is often used to guide the fitting and positioning of lenses within the frame.

Image capture data for the adjustment of spectacle lenses to a spectacle frame is determined in order to adapt the optical power of a spectacle lens, as determined by an ophthalmologist or optician on the basis of a visual acuity examination of a test person's eyes, to this spectacle frame, taking into account the physiognomy of the test person's field of vision and the geometric properties of the spectacle frame selected by the test person, in such a manner that the vision-correcting effect of the spectacle lens can be optimally utilized for the test person in the main direction of vision.

### OVERVIEW OF RELATED ART

The following discussion of related art is provided for background purposes only and is not to be construed as an admission that any of the references or techniques described constitute prior art under 35 U.S.C. § 102 or otherwise.

Methods for the mobile acquisition of image capture data for adjusting spectacle lenses to a spectacle frame by using a mobile, computer-based terminal device with touchscreen monitor are well known. For example, a video centering system for determining centering data for spectacle lenses is known, which consists of at least one image capturing device, an image processing unit with a computer and an operating unit, with all components being integrated in a mobile housing. The image capturing device can be aligned with an object in the room at a defined distance. Means for displaying visual signals are arranged on the front of the housing, and a screen for displaying the image of the object captured by the image capturing device is arranged on the rear of the housing.

The video centering system has at least one position and acceleration sensor (DE 10 2011 009 646 A1).

A handheld instrument for aligning a lens with a patient's eye is also known. The device comprises a capturing apparatus for capturing and storing an image of a patient wearing glasses, a processor for determining the center of the patient's pupil in the image and for displaying the position of the lens above the patient's eye on a display, wherein the optical center of the lens is aligned with the patient's pupil (US 2010/0220285 A1).

There is also a known method for measuring at least one geomorphometric parameter of a person wearing glasses, wherein the method comprises a stand-alone computer device comprising: a monitor, a target, a compact image capturing system comprising a means for determining its inclination that is connected to the monitor, and a processing unit permitting to control the image capturing system and to process the images obtained. The method includes the steps of: Adjusting the inclination of the image capturing system so that it is within an acceptable angular range; adjusting the height of the image capturing system so that the person can see the target along a substantially horizontal line of sight; observing the target by the person in a comfortable position without positional restrictions; capturing at least one image of the eyes or the person and the spectacle frame in this observation position; processing the at least one image using the processing unit to determine each geomorphometric parameter of the person based on the position of their pupils and the inclination of their spectacles; and returning the result of these measurements (US 2014/0240664 A1).

A method for determining a person's visual parameters is also known and comprises the following steps: Taking two pictures from two different angles of the person's face with a camera, with the person in a natural distance viewing position, determining the inclination of the camera relative to the ground at the time the pictures are taken, and deriving the inclination and direction of the person's face therefrom. The camera is positioned outside the person's foveal area when pictures are taken (US 2015/0339511 A1).

A method, device and computer program for determining at least one optical parameter of a spectacle lens are also known, wherein in the method an image is captured using the spectacle lens and at least one optical parameter of the spectacle lens is determined by means of image processing of the image, wherein the image comprises the eyes including the eye area and/or a facial area adjacent to the eyes of a test person. The device comprises at least one camera, possibly also a stereo camera, which is set up to capture an image using a spectacle lens and captures the image as described in the method. The device further comprises an evaluation unit which is set up to determine at least one optical parameter of a spectacle lens by means of image processing of the image, and, if necessary, also means for determining the distance between the camera and the user's eye (EP 3 730 998 A1). The method and device are used to determine at least one optical parameter of a spectacle lens, for which purpose at least one spectacle lens must be used. This means that it is not suitable for the process of fitting spectacle lenses into spectacle frames, which precedes the manufacture of spectacle lenses, nor for the process of manufacturing spectacle lenses themselves, as this requires image capture data that is determined by the physiognomy of the test person and the spectacle frames they have chosen.

A computer-implemented method for measuring at least one optical parameter needed to adjust a spectacle frame to a wearer is also known, in which a mobile device is used to take images of a wearer wearing a spectacle frame from different angles. At least one outline of the spectacle frame is captured from the images, from which at least one adjustment parameter of the spectacle frame is derived. Said outline is determined using artificial intelligence (EP 4 249 994 A1). The disadvantage of this method is that several photographs must be taken from different angles of the camera relative to the test person, which makes the procedure relatively time-consuming and exposes the test person to a certain amount of physical strain due to the duration of the procedure.

### SUMMARY

Disclosed herein is a method for determining at least one parameter in connection with an ophthalmic device is known which uses at least two cameras arranged in the same vertical plane at a horizontal distance from each other. The determination is made on the basis of a photograph of the face of the person wearing a selected spectacle frame. The second camera enables a three-dimensional representation of the scene, also known as a stereoscopic image, to be obtained after processing the images received from the main camera. The method comprises the steps of:
1. Capturing at least one characteristic point of at least one eye of the person captured in the image and estimating the three-dimensional position of the captured characteristic point(s);
2. Recognizing the worn spectacle frame and estimating the three-dimensional position of the worn frame by aligning a three-dimensional representation of the worn frame with the worn frame in the captured image; and
3. Determining one or more parameters from the relative position of the eyes in relation to the three-dimensional representation of the selected spectacle frame (U.S. Pat. No. 11,158,082 B2).

In this procedure, the image capture data is determined using a reference spectacle frame, which not only means a detour, but also requires the test person to remain in the same position for a longer period of time, which is practically impossible for the test person, or at least involves a high degree of physical strain. Even the slightest deviation from the initial position of the head represents a significant source of measurement error. Since it is practically impossible for the test person to maintain exactly the same positions for the reference spectacle frame and the spectacle frame to be measured in the 3D space of the cameras, the measurement results are typically inaccurate.

Computer-assisted stereo camera systems are also known, in which the image capture (shot) is triggered by the operator via a computer function or is triggered automatically by the computer itself, with the cameras receiving a trigger command (shot) via one or two interfaces of the computer. It is also known that electronic cameras are equipped with their own clock functions, for example with quartz clock generators. Both technical solutions have the disadvantage that the two images captured by the individual cameras are not completely synchronized. For example, when triggering image capture (the shot command) via a computer's I2C bus, there is a time delay of 12-15 ms between the individual cameras. In addition, the clock generators of the individual cameras are not synchronized and therefore cannot take pictures at exactly the same time. This is to say that when capturing moving objects, such as persons, the two cameras do not record the exact same position, and the subsequent stereoscopic measurement can lead to measurement errors.

The object of this disclosure is therefore to create a method for the mobile acquisition of image capture data for spectacle lenses that are to be fitted into spectacle frames, which, compared to the prior art, requires less technical effort and time and thus also causes less physical strain on the test person, while ensuring a high degree of accuracy and reliability of the image capture data. A further object of the disclosure is to employ conventional mobile PC technology in order to create an easy-to-use mobile capturing system for acquiring image capture data for spectacle lenses that are to be fitted into a spectacle frame, while avoiding the measurement errors inherent in prior art devices.

The method according to the disclosure has the advantage that it does not require any auxiliary elements that serve as a reference, such as sighting frames or reference spectacle frames. Only a frontal image of the test person is required, so that the burden on the test person can be kept within reasonable limits.

According to the disclosure, this is achieved by using a mobile computer-based terminal device with touchscreen and a stereo camera system comprising at least two electronic cameras spaced apart from each other to photographically capture the facial area of a test person wearing an anatomically well-fitting spectacle frame, wherein the at least two cameras simultaneously generate synchronous images of the test person in such a manner that they are triggered clock-synchronously by means of the mobile computer-based terminal device using a clock signal and an image trigger signal or a clock signal, an image trigger signal and a flash trigger signal. Thus, it is possible to create technically identical images of the face of the test person.

In an advantageous configuration of the disclosure, the at least two individual cameras controlled by a minicomputer are positioned vertically below each other so that the synchronous images are displayed vertically one above the other on the touchscreen.

In an additional advantageous configuration of the disclosure, the trigger process causes the two portrait shots to be displayed as synchronous still images on the respective image halves of the touchscreen monitor's image area, approximately in a center position.

In another advantageous configuration of the disclosure, the two images are created with the cameras in a horizontal orientation.

In another advantageous configuration, a spirit level is displayed in the overall live image on the touchscreen monitor, by means of which the operator aligns the optical axes of the at least two cameras in the zero viewing direction of the test person. The virtual spirit level makes it easier for the operator to align the system so that the optical axes of the two cameras are oriented horizontally, allowing both portrait photographs of the test person to be taken with their gaze directed in a zero viewing direction, i.e. looking freely straight ahead. By manually triggering the cameras on the touchscreen or by computer-controlled automatic triggering, it is thus possible to generate synchronous still images on the respective image halves as symmetrically superimposed portrait shots.

In another advantageous configuration, a three-dimensional spirit level is displayed in the overall live image on the touchscreen monitor, by means of which the operator can additionally align the optical axes of the at least two cameras in a vertical and/or horizontal position. This allows second-order errors in distance measurement to be compensated for.

In another advantageous configuration of the disclosure, the still images of the test person are evaluated by means of stereo image evaluation.

The mobile capturing system according to this disclosure for acquiring image capture data for spectacle lenses needed to adjust the spectacle lenses to a spectacle frame consists of a mobile computer-based terminal device with touchscreen monitor and of a stereo camera system comprising at least two electronic cameras spaced apart from each other. The mobile computer-based terminal device has a synchronization unit which provides a clock signal and an image trigger signal or a clock signal, an image trigger signal and a flash trigger signal in clock-synchronous timing for triggering the at least two electronic cameras.

The mobile capturing system according to the disclosure is easy to use and, by resorting to stereo image evaluation, enables high accuracy even in the viewing direction of the cameras, the so-called depth values in the direction of the z-axis of the camera. Furthermore, measurement errors caused by a time delay in the stereo camera system's capturing are avoided by a clock-synchronous capturing technique implemented in the mobile capturing system's computer.

In an advantageous configuration of the disclosure, the at least two spaced-apart electronic cameras are arranged one above the other in a vertical position at the time of capturing, with the optical axis of each camera being aligned in a horizontal direction during capturing. To make it easier for the operator to align the optical axis of each camera when capturing in the zero viewing direction of the test person, a spirit level, preferably a three-dimensional spirit level, is displayed in the overall live image on the touchscreen monitor, by means of which the operator can additionally align the optical axes of the at least two cameras in a vertical and/or horizontal position.

Further advantages and advantageous configurations of the disclosure may be found in the following description, in the claims, and in the drawings.

### BRIEF DESCRIPTION OF DRAWINGS

A preferred embodiment example of a mobile capturing system is represented in the drawings and is discussed in greater detail in the following. In the drawings:

FIG. 1 is a schematic diagram of a mobile capturing system, and

FIG. 2 is block diagram of the mobile capturing system.

### DETAILED DESCRIPTION

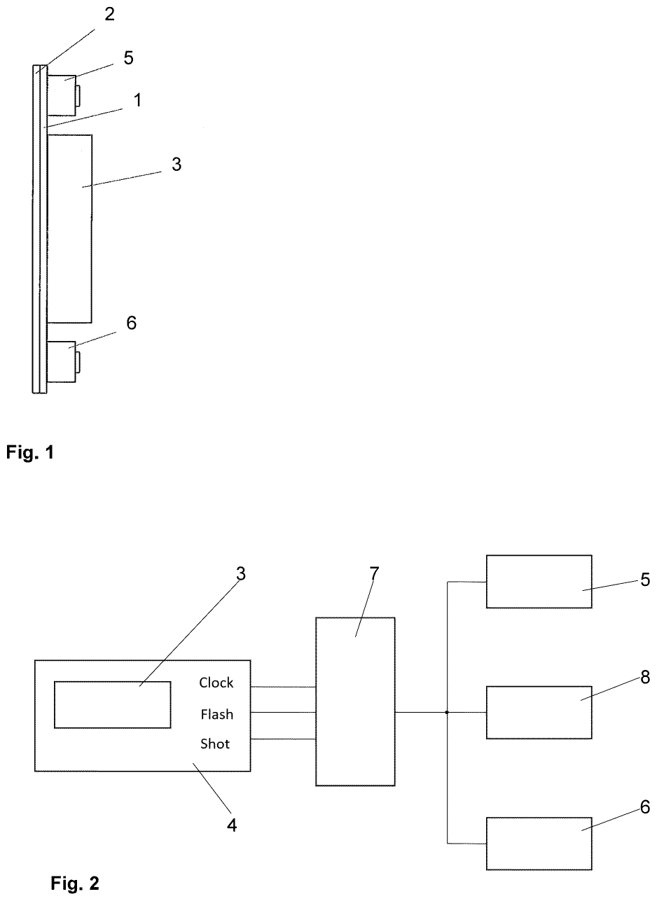

As can be seen in FIG. 1, the mobile capturing system has a chassis 1 having a touchscreen 2 disposed on the side facing the operator. The side facing the person to be photographed has a minicomputer 3 with its main system board, i.e. its motherboard 4, as well as an upper camera 5 and a lower camera 6 arranged thereon.

FIG. 2 shows a block diagram of a mobile capturing system according to the disclosure. The mainboard 4 of the minicomputer 3 is connected to a synchronization unit 7, which controls the two cameras 5, 6 and a flash 8 of the capturing system. Via its system motherboard, i.e. its mainboard 4, the minicomputer 3 provides the synchronization unit 7 with a clock signal (Clock), an image trigger signal (Shot) and, if necessary, a flash trigger signal (Flash). The synchronization unit 7 contains pure hardware solutions that transmit the trigger signals (Shot, Flash) to both cameras 5, 6 and, if necessary, to the flash 8 simultaneously and without delay. Since the two cameras 5, 6 and, if applicable, the flash 8 are controlled and triggered clock-synchronously via the central clock signal (Clock), the processing, i.e. the capturing itself, is also absolutely simultaneous (synchronous) in both cameras 5, 6. Thus, the images produced in this way form the basis for an error-free acquisition of the image capture data.

Various features described herein may be implemented in combination or independently, as appropriate to the implementation context.
1 chassis
2 touchscreen
3 computer
4 mainboard
5 upper camera
6 lower camera
7 synchronization unit
8 flash

What is claimed is:

1. A method for mobile acquisition of image capture data for purpose of adapting spectacle lenses to a spectacle frame using a mobile computer-based terminal device with a touchscreen monitor and a stereo camera system comprising at least two spaced-apart electronic cameras that photographically captures a facial area wearing an anatomically well-fitted spectacle frame, the method comprising:
generating simultaneously by the at least two spaced-apart electronic cameras synchronous images of the facial area in such a manner that they are triggered clock-synchronously by means of the mobile computer-based terminal device using a clock signal and an image trigger signal or a clock signal, an image trigger signal and a flash trigger signal.

2. The method as claimed in claim 1,
further comprising:
directing a mobile terminal device towards the facial area in such a manner that the at least two spaced-apart electronic cameras are positioned vertically one above the other, so that the synchronous images are displayed vertically one above the other on the touchscreen monitor.

3. The method as claimed in claim 1,
wherein:
a trigger process causes two portrait shots to be displayed as synchronous still images on respective halves image area of the touchscreen monitor approximately in a centre position.

4. The method as claimed in claim 1,
wherein:
the two synchronous images are created with the at least two spaced-apart electronic cameras in a horizontal orientation.

5. The method as claimed in claim 1,
further comprising:
displaying a spirit level an overall live image on the touchscreen monitor, by means of which an operator aligns an optical axes of the at least two spaced-apart electronic cameras in a zero viewing direction of the facial area.

6. The method as claimed in claim 5,
wherein:
a three-dimensional spirit level is displayed in the overall live image on the touchscreen monitor, by means of which the operator additionally aligns the optical axes of the at least two spaced-apart electronic cameras in a vertical and/or horizontal position.

7. The method as claimed in claim 1,
further comprising:
evaluating still images of the facial area of stereo image evaluation.

8. A mobile capturing system for acquiring image capture data for spectacle lenses configured to adjust the spectacle lenses to a spectacle frame, comprising a mobile computer-based terminal device with a touchscreen monitor and a stereo camera system having at least two electronic cameras spaced apart from each other,
wherein:
the mobile computer-based terminal device has a synchronisation unit which provides a clock signal and an image trigger signal or a clock signal, an image trigger signal and a flash trigger signal in clock-synchronous timing for triggering the at least two electronic cameras.

9. The mobile capturing system as claimed in claim 8,
wherein:
the at least two spaced-apart electronic cameras are arranged one above the other in a vertical position at a time of capturing.

10. The mobile capturing system as claimed in claim 8,
wherein:
an optical axis of each camera is aligned in a horizontal direction during recording.

11. The mobile capturing system as claimed in claim 8,
wherein:
a spirit level is displayed in an overall live image on the touchscreen monitor.

12. The mobile capturing system as claimed in claim 8,
wherein:
a sighting mark is arranged centrally between the at least two spaced-apart electronic cameras.
